(19) United States
(12) Plant Patent Application Publication
(10) Pub. No.: US 2002/0073460 P1
(43) Pub. Date: Jun. 13, 2002

(54) ALSTROEMERIA PLANT NAMED "STABECOR"

(76) Inventor: C. A. HOOGENDOORN, NIEUWKOOP (NL)

Correspondence Address: CHRISTIE PARKER & HALE, P O BOX 7068, PASADENA, CA, 911097068

(21) Appl. No.: 09/349,613

(22) Filed: Jul. 8, 1999

(51) Int. Cl. A01H 5/00

(52) U.S. Cl. PLT/309

(57) ABSTRACT

A new and distinct Alstroemeria plant variety producing flowers of good quality and attractive coloration.

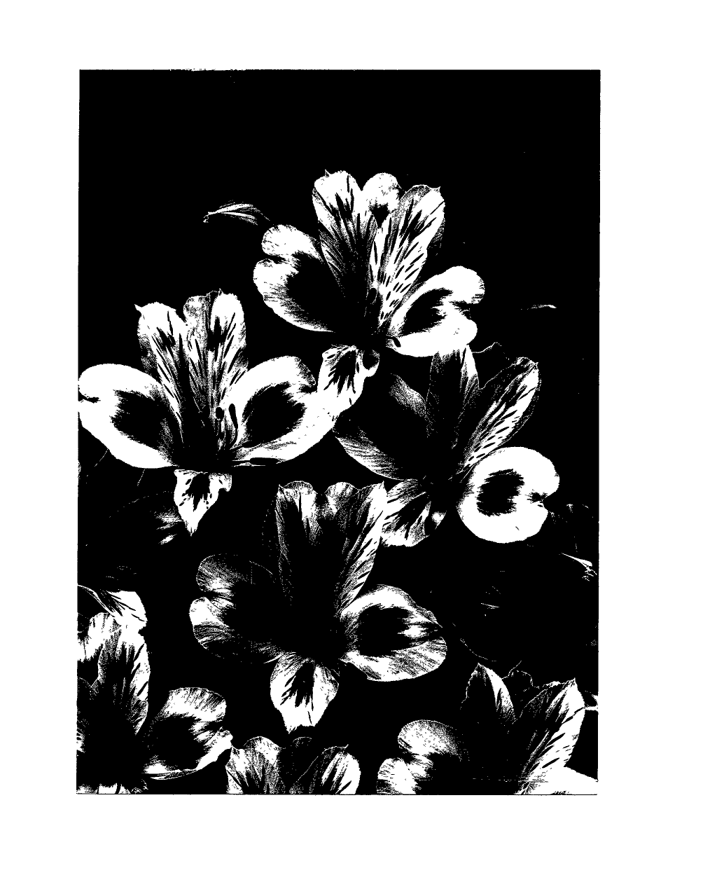

### BACKGROUND OF THE INVENTION

The present invention relates to a new and distinct variety of Alstroemeria which is a sport of the variety ‘Stabec’ discovered in a controlled planting. The varietal denomination of the new variety is ‘Stabecor’.

The first act of asexual reproduction of ‘Stabecor’ was in Aalsmeer, The Netherlands, by propagation by dividing the rhizomes. Asexual reproduction by dividing rhizomes through successive generations in Aalsmeer, The Netherlands, has demonstrated that the combination of characteristics as herein disclosed for ‘Stabecor’ are firmly fixed and retained through successive generations.

### SUMMARY OF THE INVENTION

The new variety of Alstroemeria, ‘Stabecor’, produces large flowers of good quality.

### COMPARISON WITH PARENT

The new variety produces flowers of different color than its parent.

### DESCRIPTION OF ILLUSTRATION

The new variety of Alstroemeria hybrid is illustrated in the accompanying illustration, which shows typical flower characteristics, with colors being as nearly true as is possible for illustrations of this type.

### DETAILED DESCRIPTION OF THE NEW VARIETY

The following is a detailed description of the new cultivar of Alstroemeria hybrid as observed in a greenhouse in Aalsmeer, The Netherlands. Color designations indicated are in accordance with The Royal Horticultural Society Color Chart by color plate designations.

‘Stabecor’ has not been observed under all possible environmental conditions. Phenotypic expression may vary with variations in environment such as temperature, light intensity, day length and growing and cultural conditions.

Botanical classification: Family: Amaryllidaceae Genus: Alstroemeria sp.

Plant:

| | |
|---|---|
| Form: | Bush |
| Shape: | Upright |
| Growth: | Fast |
| Height: | About 150-180 cm |
| Main stem: | About 170 cm |
| Internode: | About 1-8 cm |
| Branching | |
| Characteristics | |
| Growth: | Vigorous, strong |
| Foliage | |
| Quantity: | Abundant |
| Number of leaves: | About 35-45 |
| Size: | About 15-20 cm long; about |
| | 3-5 cm wide |
| Shape: | Elliptical |
| Margin: | Minutely crimped |
| Texture: | Glossy |
| Color | |
| Upper side: | Near 137A |
| Underside: | Near 137C |
| Rhizomes: | About 10-30 cm, near 155D |

Inflorescence:

| | |
|---|---|
| Blooming habit: | Continuous; blooms |
| | profusely in Spring and |
| | Fall |
| Size: | About 7 cm in depth; about |
| | 5 cm in diameter when |
| | anthers release their |
| | pollen |
| Borne: | Singly |
| Shape: | Cup when bloom first opens |
| | and matures |
| Petalage | |
| Number of tepals: | 6 |
| Arrangement: | Two concentric circles of |
| | 3 tepals |
| Form: | Obovate |
| Margin type: | Minutely crimped |
| Apex: | Pointed |
| Tepals: | Stay on stem about 4 weeks |
| Texture: | Soft |
| Appearance: | Velvety |
| Color: | |
| Outer tepal: | Body near 29B to 51A; base |
| | near 62D; underside near |
| | 29B to 51A |
| Inside tepal: | Body near 6A to 51A; base |
| | near 62D; underside near |
| | 6A to 51A; two inner |
| | lateral tepals have about |
| | 20-40 spots near 185A; |
| | inner medial tepal has |
| | about 0-15 spots near 185A |
| Peduncle | |
| Length: | About 10-15 cm |
| Color: | Near 137B |
| Strength: | Erect, Strong, upright |
| Hairy: | No |
| Discoloration: | None |
| Bloom affected by | |
| hot or wet weather: | No |
| Disease resistance: | Unknown |
| Fragrance: | None |
| Lasting quality: | On plant about 4 weeks; as |
| | cut flower about 18-20 |
| | days |
| Form: | Pear-shaped, not pointed |
| Color of tepals: | Near 51A when tepals first |
| | divide; near 51A when |
| | tepals begin to unfurl |

Reproductive organs:

| | |
|---|---|
| Stamen: | About 6 |
| Arrangement: | Side-by-side around style |
| Anthers: | About 8-10 mm |
| Color: | Near 161A |
| Filaments: | About 5 cm |
| Color: | Near 51A |
| Pollen: | Near 6C |
| Pistils: | 1 |
| Styles: | About 4-5 cm |
| Color: | Near 51A |
| Stigma: | Near 51A |
| Fertility: | Fertile |
| FRUIT: | Round, near 163A |

I claim:

1. A new and distinct Alstroemeria plant of the variety substantially as shown and described.
